(19) United States
(12) Patent Application Publication
(10) Pub. No.: US 2022/0295977 A1
(43) Pub. Date: Sep. 22, 2022

(54) BRUSH WITH LED LIGHT SOURCE

(71) Applicant: Japan Display Inc., Tokyo (JP)

(72) Inventor: Masanobu IKEDA, Tokyo (JP)

(73) Assignee: Japan Display Inc., Tokyo (JP)

(21) Appl. No.: 17/696,924

(22) Filed: Mar. 17, 2022

(30) Foreign Application Priority Data
Mar. 19, 2021 (JP) 2021-046458

(51) Int. Cl. A46B 15/00; A46B 7/04

(52) U.S. Cl. CPC A46B 15/0036; A46B 7/04; A46B 15/0034; A46B 2200/1066

(57) ABSTRACT

A brush with an LED light source includes a body, and a head provided at a tip of the body, the head including a housing part connected to the body, a support part detachably attached to the housing part, a brush part formed of a plurality of bristles planted in the support part, and a light-source part provided inside the housing part, wherein the light-source part has a first LED for emitting light having a peak in a first wavelength band and a second LED for emitting light having a peak in a second wavelength band different from the first wavelength band.

### CROSS-REFERENCE TO RELATED APPLICATION

This application is based upon and claims the benefit of priority from the prior Japanese Patent Application No. 2021-046458 filed on Mar. 19, 2021, the entire contents of which are incorporated herein by reference.

### FIELD

An embodiment of the present invention relates to a brush with an LED light source.

### BACKGROUND

Toothbrushes that perform tooth brushing by applying a brush that vibrates at high speed to the teeth are known. In recent years, toothbrushes that not only vibrate at high speed but also enhance the sterilizing effect and promote blood circulation in gums by applying light emitted from an LED to teeth and gums have been developed.

Patent Literature 1 discloses a toothbrush in which an ultraviolet irradiation part is provided at a head (WO 2017/213017). Patent Literature 2 discloses an electric toothbrush in which light emitted from an LED provided in a handle part is reflected by a mirror provided in a head to emit light from a bristle bundle of the brush (Japanese Laid-Open Patent Publication No. 2006-110085). The blood circulation of the gums can be promoted by heating the gums by light emitted from the bristle bundle of the brush.

### SUMMARY

A brush with an LED light source according to an embodiment of the present invention includes a body, and a head provided at a tip of the body, the head including a housing part connected to the body, a support part detachably attached to the housing part, a brush part formed of a plurality of bristles planted in the support part, and a light-source part provided inside the housing part, wherein the light-source part has a first LED for emitting light having a peak in a first wavelength band and a second LED for emitting light having a peak in a second wavelength band different from the first wavelength band.

### BRIEF DESCRIPTION OF DRAWINGS

FIG. 1 is a cross-sectional view of a brush with an LED light source according to an embodiment of the present invention;

FIG. 2 is a diagram illustrating an outline of a brush with an LED light source according to an embodiment of the present invention;

FIG. 3 is a plan view when a support part in a head of a brush with an LED light source according to an embodiment of the present invention is viewed from above;

FIG. 4 is a plan view when a light-source part arranged in a housing part of a brush with an LED light source according to an embodiment of the present invention is viewed from above;

FIG. 5 is a cross-sectional view when a brush with an LED light source is shown in FIG. 3 cut along a line A1-A2;

FIG. 6 is a block diagram showing a hardware configuration of a brush with an LED light source;

FIG. 7 is an example of a light-source part;

FIG. 8 is an example of a pixel circuit in a pixel;

FIG. 9 is a timing chart of a light-emitting pattern of a light-emitting element;

FIG. 10 is a cross-sectional view of a brush with an LED light source according to an embodiment of the present invention;

FIG. 11 is an example of a light-source part;

FIG. 12 is a cross-sectional view of a head of a brush with an LED light source according to an embodiment of the present invention;

FIG. 13 is a cross-sectional view of a head of a brush with an LED light source according to an embodiment of the present invention; and

FIG. 14 is a cross-sectional view of a head of a brush with an LED light source according to an embodiment of the present invention.

### DESCRIPTION OF EMBODIMENTS

In a conventional toothbrush with an LED, when the bristles of the brush deteriorate or the head part is scratched, the handle part and the head part are integrally formed, so that the head part is difficult to replace.

One object of the present invention is to provide a brush with an LED light source in which the head portion can be easily replaced.

Embodiments of the present invention will be described below with reference to the drawings and the like. However, the present invention can be implemented in various modes without departing from the gist thereof and should not be construed as being limited to the description of the following embodiments. With reference to the drawings, although the width, thickness, shape, and the like of each part may be schematically represented as compared with actual embodiments to make the description clearer, the schematic drawings are only examples and do not limit the interpretation of the present invention. In addition, in the present specification and each of the drawings, the same or similar elements as those described with reference to the preceding drawings are denoted by the same symbols, and a repetitive description thereof may be omitted.

In this specification, expressions such as “above”, “below” and the like when describing the drawings represent relative positional relationships between a structure of interest and other structures. In this specification, in a side view, a direction from an insulating surface to a light-emitting element, which will be described later, is defined as “above”, and the opposite direction is defined as “below”. In this specification and claims, when expressing the mode of arranging another structure on a certain structure, both the case of arranging another structure directly above a certain structure and the case of arranging another structure above a certain structure via another structure are included, unless otherwise specified.

### First Embodiment

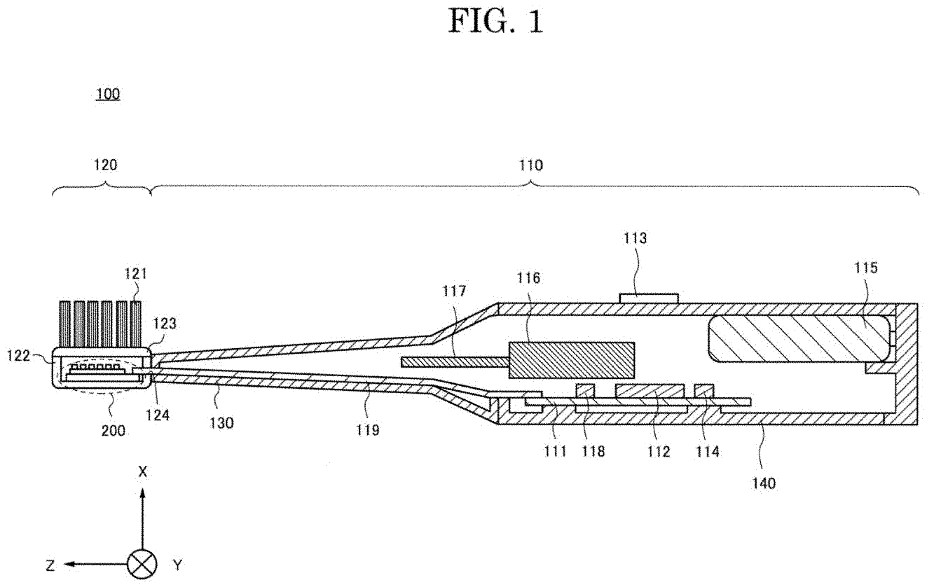

A brush with an LED light source 100 according to an embodiment of the present invention will be described with reference to FIG. 1 to FIG. 10.

<Structure of Brush with an LED Light Source>

FIG. 1 is a schematic diagram illustrating a structure of the brush with an LED light source 100 according to an embodiment of the present invention. The brush with an LED light source 100 includes a body 110 and a head 120 provided on a tip of the body 110. In the following description, the direction along the extending direction of the brush with an LED light source 100, which is generally axial, is defined as the Z-direction. Within a plane perpendicular to the Z-direction, the X-direction and Y-direction perpendicular to each other are defined. Of these, the X-direction is a direction perpendicular to a surface on which a brush part 121 in the head 120 is formed.

The brush with an LED light source 100 according to an embodiment of the present invention is an electric toothbrush that automatically performs a tooth brushing operation and its auxiliary operation by a turning operation of the head 120 including the brush part 121 to a handle part 140 that is gripped by a user.

The brush with an LED light source 100 includes the body 110 and the head 120. The body 110 includes a neck part 130 and the handle part 140. In the neck part 130, one end in the Z-direction is connected to the head 120, and the other is connected to the handle part 140. In the neck part 130, a part connected to the head 120 is a part that reaches into the oral cavity of the user at the time of brushing teeth. A material of the handle part 140 and the neck part 130 is preferably, for example, a resin.

The handle part 140 is a part that is gripped by a user when brushing teeth. The handle part 140 is provided with a control substrate 111, a controller 112, an operation part 113, a battery 115, a sensor part 114, and a driver 116.

The controller 112, the operation part 113, the sensor part 114, the battery 115, the driver 116, and an LED driver 118 are electrically connected, respectively, on the control substrate 111. The battery 115 provides a power source to various components by the control substrate 111.

The controller 112 is, for example, a micro-controller. The operation part 113 is a pushbutton for turning the brush with an LED light source 100 on and off and to change operation modes of the toothbrush according to the user's preference. The operation modes will be described in detail later. The sensor part 114 is, for example, an acceleration sensor for detecting the inclination of the brush with an LED light source 100. The driver 116 is a power generating part that provides vibrations to the neck part 130 for tooth brushing. The driver 116 includes, for example, a motor shaft 117 extending in the Z-direction. A linear actuator that vibrates the motor shaft 117 with the Z-direction as the vibration direction can be used as the driver 116.

The control substrate 111 is provided with the LED driver 118. The LED driver 118 is electrically connected to a light-source part 200 via an FPC 119. The FPC 119 supplies a signal output from the LED driver 118 to the light-source part 200.

The head 120 includes a housing part 122, a support part 123, the brush part 121, and the light-source part 200. The light-source part 200 is provided in the housing part 122. The housing part 122 is connected to one end of the neck part 130. In the housing part 122, an opening 124 is provided at a connection part with the neck part 130. The FPC 119 extending from the neck part 130 is inserted into the opening 124 of the housing part 122 and connected to the light-source part 200. The support part 123 is connected to the housing part 122. The support part 123 is planted with the brush part 121 formed of a plurality of bristle bundles.

<Overview of Brush with LED Light Source>

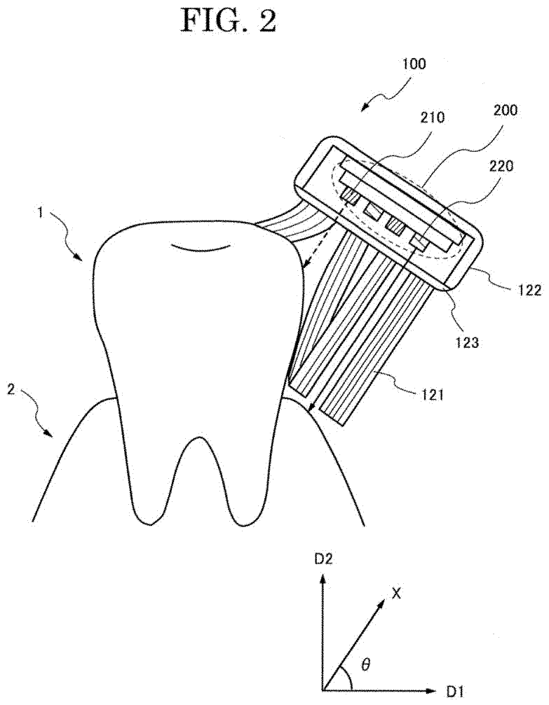

FIG. 2 is a diagram illustrating an outline of the brush with an LED light source 100 according to an embodiment of the present invention. FIG. 2 shows the head 120 of the brush with an LED light source 100 abutting against teeth 1 and gum 2. The light-source part 200 provided in the housing part 122 and the brush part 121 planted in the support part 123 are provided in the head 120 of the brush with an LED light source 100.

A light-emitting element 210 and a light-emitting element 220 for emitting light having emission peaks in different wavelength regions from each other are arranged in a matrix in the light-source part 200. The light-emitting element 210 and the light-emitting element 220 are, for example, LEDs (Light Emitting Diode). In the present embodiment, the light-emitting element 210 has a peak in the wavelength band of 315 nm to 400 nm, and the light-emitting element 220 has a peak in the wavelength band of 610 nm to 780 nm. In other words, the light-emitting element 210 emits ultraviolet light and the light-emitting element 220 emits red light.

When the brush with an LED light source 100 is turned on, the motor shaft 117 starts to vibrate by the driver 116. At the same time, in the light-source part 200, two types of the light-emitting element 210 and the light-emitting element 220 start to emit light. When brushing the teeth 1 with the brush with an LED light source 100, the brush part 121 of the brush with an LED light source 100 abuts the teeth 1 and the gum 2. At this time, the light-emitting element 210 and the light-emitting element 220 of the light-source part 200 provided inside the head 120 are emitting light. For example, the light emitted from the light-emitting element 220 is irradiated to the gum 2, and the light emitted from the light-emitting element 210 is irradiated to the teeth 1. The light emitted from the light-emitting element 210 is ultraviolet light. By applying ultraviolet light to the teeth 1, plaque attached to the teeth 1 can be decomposed. The light emitted from the light-emitting element 220 is red light. By applying red light to the gum 2, the gum 2 is warmed so that blood circulation in the gum 2 is promoted.

The brush with an LED light source 100 allows the decomposition of plaque by ultraviolet and the promotion of blood circulation in the gum 2 by red light, thus providing efficient oral care.

The brush with an LED light source 100 according to an embodiment of the present invention can set a light-emitting pattern of the light-emitting element 210 and a light-emitting pattern of the light-emitting element 220, respectively. This allows the brush with an LED light source 100 to make the light-emitting element 210 emit light and irradiate only the ultraviolet light to the teeth 1, make the light-emitting element 220 emit light and irradiate only the red light to the gum 2, or irradiate both the ultraviolet light and the red light to the teeth 1 and the gum 2. When detecting the inclination of the head 120 by the sensor part 114, the light-emitting pattern of the light-emitting element 220 and the light-emitting pattern of the light-emitting element 210 may be changed according to the inclination of the head 120. For example, in FIG. 2, in the case where the inclination 8 of the head 120 in the first direction D1 is large, the light-emitting element 210 may be made to emit light and only the ultraviolet light may be applied to the teeth 1. Alternatively, in the case where the inclination 8 of the head 120 in the first direction D2 is small, the light-emitting element 220 may be made to emit light and only the red light may be applied to the gum 2. In this case, the inclination of the head 120 refers to the inclination of the X-direction with respect to the first direction D1 in the brush with an LED light source 100.

<Structure of Head>

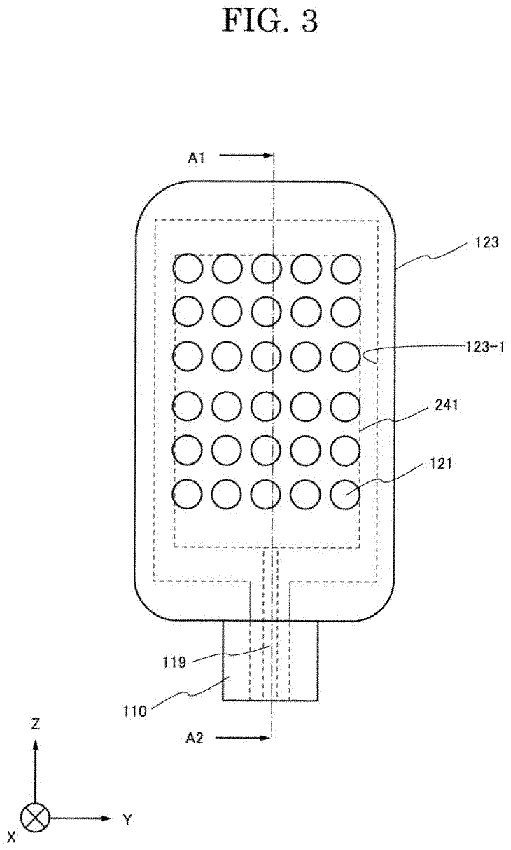

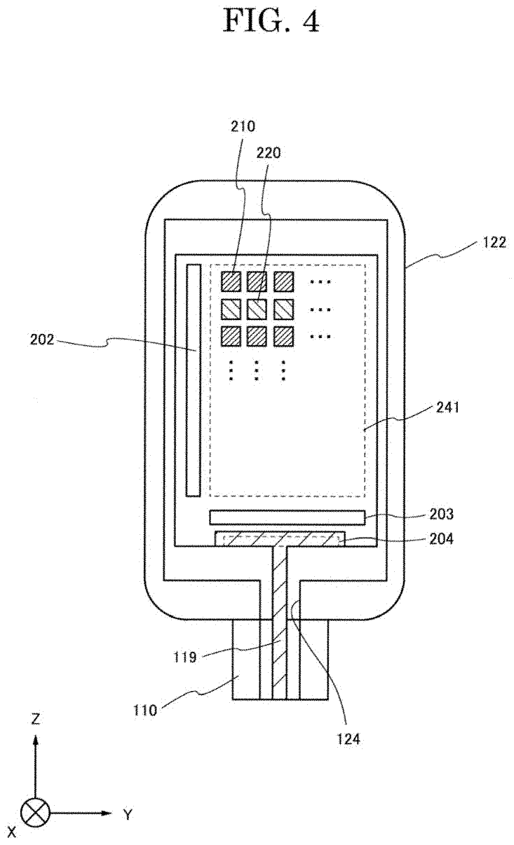

Next, a structure of the head 120 of the brush with an LED light source 100 will be described in detail with reference to FIG. 3 to FIG. 5. FIG. 3 is a plan view when the support part 123 of the head 120 of the brush with an LED light source 100 is viewed from above. FIG. 4 is a plan view when the light-source part 200 arranged in the housing part 122 is viewed from above.

As shown in FIG. 3, the brush part 121 having the plurality of bristle bundles is regularly arranged on the support part 123. The number of the brush parts 121 and the position where the brush part 121 is arranged are not particularly limited. The support part 123 is connected to the housing part 122. An inner wall 123-1 of the housing part 122 is shown by a dotted line. The light-source part 200 is provided inside the housing part 122. The light-source part 200 is provided overlapping the brush part 121. A part of the housing part 122 is connected to the body 110.

As shown in FIG. 4, the light-source part 200 is provided in the housing part 122. The housing part 122 and the body 110 are provided with the opening 124 for inserting the FPC 119. The FPC 119 extending from the control substrate 111 in the body unit 110 is connected to the light-source part 200.

The light-source part 200 is provided with a light-emitting part 201 having the light-emitting element 210 and the light-emitting element 220. The light-source part 200 is provided with a scan line drive circuit 202 and a signal line drive circuit 203 for controlling the light-emitting part 201. The scan line drive circuit 202 and the signal line drive circuit 203 are supplied with a signal from the LED driver 118 via the FPC 119. The light-emitting element 210 and the light-emitting element 220 are controlled to emit light by a signal supplied from the scan line drive circuit 202 and the signal line drive circuit 203.

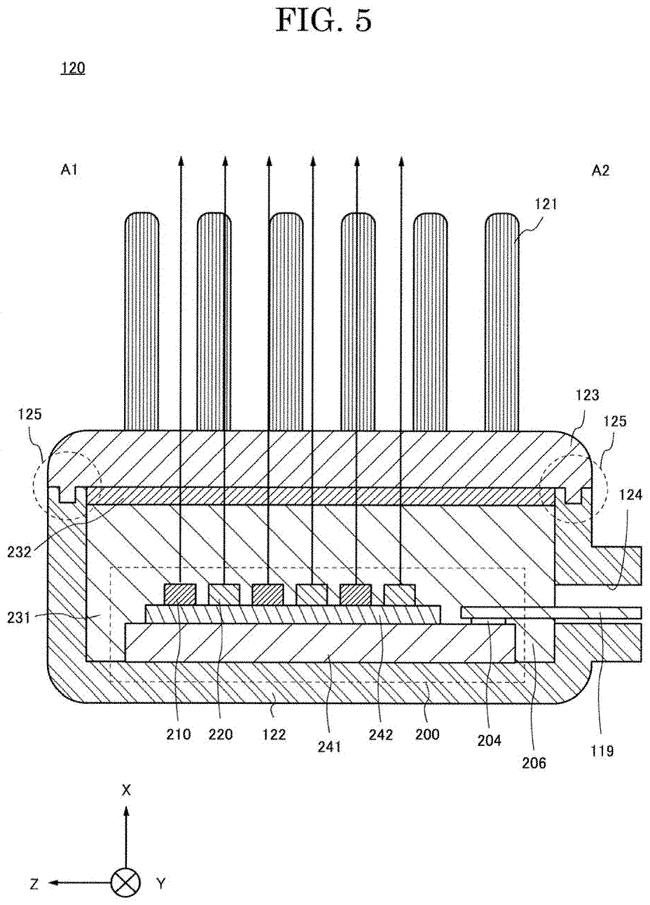

FIG. 5 is a cross-sectional view in which the head 120 shown in FIG. 3 is cut along a line A1-A2. The light-source part 200 is provided inside the housing part 122. The light-source part 200 includes a substrate 241, an element forming layer 242 provided on the substrate 241, and the light-emitting element 210 and the light-emitting element 220 provided on the element forming layer 242. For example, a glass substrate, a plastic substrate, a silicone substrate, a sapphire substrate, or the like is used as the substrate 241. The element forming layer 242 is provided with a plurality of transistors. The light-emitting element 210 and the light-emitting element 220 are provided on the element forming layer 242.

A terminal part 204 is provided at an end of the substrate 241. The terminal part 204 is connected to the FPC 119. The terminal part 204 supplies a signal to the transistors via a wiring formed in the element forming layer 242. The light-emitting element 210 is controlled to be turned on and off by the signal supplied to the transistors. Similarly, the light-emitting element 220 is controlled to be turned on and off by the signal supplied to the transistors.

The inside of the housing part 122 is filled by a resin layer 231 having light transmittance. Examples of the resin layer 231 having light transmittance include an epoxy resin and an acrylic resin. By filling the inside of the housing part 122 with the resin layer 231 having light transmittance, it is possible to suppress moisture from entering into the housing part 122. By using the resin layer 231 having light transmittance, the light emitted from the light-emitting element 210 and the light-emitting element 220 can be easily extracted to the outside.

The support part 123 is also preferably formed of a resin having light transmittance. The support part 123 is flocked with the brush part 121 formed of the plurality of bristle bundles. The brush part 121 is formed of nylon, sponge, animal hair, vegetable fiber, or the like. The housing part 122 and the support part 123 are connected by a fitting part 125. For example, it is possible to attach the support part 123 to the housing part 122 by fitting a convex part provided in the support part 123 to a concave part provided in the housing part 122. Therefore, it is possible to easily remove the support part 123 from the fitting part 125 when the brush part 121 is deteriorated or the support part 123 is scratched. A new support part 123 can be easily attached to the housing part 122. In the brush with an LED light source 100, only the support part 123 needs to be replaced, and the entire head 120 provided with the light-source part 200 does not need to be replaced, thereby reducing cost.

It is preferable that a glass 232 is arranged on the upper part of the resin layer 231 in the housing part 122. The glass 232 is provided to cover the resin layer 231. Side surfaces of the glass 232 are preferably in contact with side surfaces of the housing part 122. An upper surface of the glass 232 is preferably provided to be in contact with the support part 123. That is, an upper surface of a support part 123 preferably substantially coincides with an upper surface of a wall part of the housing part 122. As a result, even if moisture enters from the gap between the housing part 122 and the support part 123, it is possible to suppress moisture from entering into the housing part 122. In addition, it is possible to suppress the resin layer 231 from being damaged when the support part 123 is attached to or detached from the housing part 122. Thus, since it is possible to suppress the light-source part 200 from deteriorating, it is possible to prolong the life of the brush with an LED light source 100.

In this case, abrasive particles contained in toothpaste contain gold or nickel, and these materials are negatively charged. In the case of using a material having a polarity opposite to the polarity of the abrasive particles, that is, a in the case where positively charged material is used as the material used for the support part 123 in the triboelectric series, the abrasive particles tend to adhere to the support part 123. Since the abrasive particles adhere to the support part 123, the light emitted from the light-emitting element 210 is refracted, and it is difficult to irradiate the light to a desired location. The epoxy resin and the acrylic resin are materials that are negatively charged in the triboelectric series. By using a material that is negatively charged in the triboelectric series as a material used for the support part 123, it is possible to suppress the abrasive particles from adhering to the support part 123. As a result, it is possible to suppress the refraction of the light emitted from the light-emitting elements 210 and 220.

<Hardware Configuration of Brush with an LED Light Source>

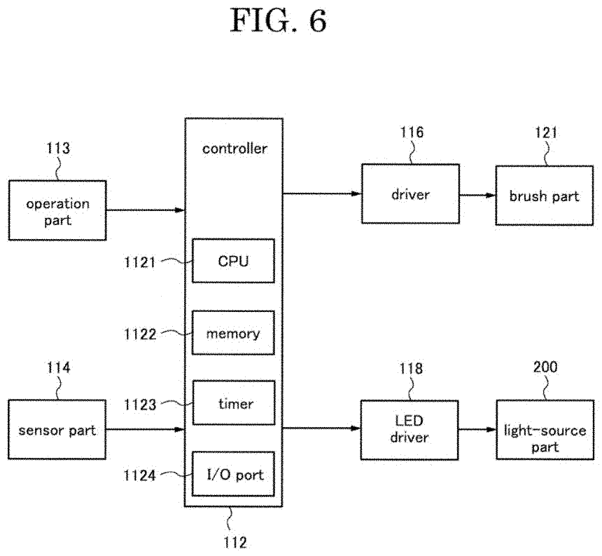

Next, a hardware configuration of the brush with an LED light source 100 will be described with reference to FIG. 6.

FIG. 6 is a block diagram showing a configuration of the brush with an LED light source 100 in an embodiment of the present invention. The brush with an LED light source 100 includes the controller 112, the operation part 113, the sensor part 114, the LED driver 118, the light-source part 200, the driver 116 (motor), and the brush part 121.

The controller 112 is, for example, a microcomputer, and includes at least a CPU 1121, a memory 1122, a timer 1123, and an I/O port 1124. The controller 112 reads out control programs stored in the memory 1122 and executes the control programs by the CPU 1121 to realize various functions in the brush with an LED light source 100. The memory 1122 includes a volatile memory and a non-volatile memory. The memory 1122 stores the control programs. The control program is, for example, an operation mode of the brush with an LED light source 100. The operation mode is the vibration pattern and the vibration speed of the driver 116, and the light-emitting pattern of the light-emitting element 210 and the light-emitting element 220 and a combination of the light-emitting patterns and the vibration pattern. When it is detected that the operation part 113 is pressed, the timer 1123 measures the time after the operation part 113 is pressed. The I/O port 1124 outputs a signal to the driver 116 and the LED driver 118 in response to a signal acquired from the operation part 113 or the sensor part 114.

The operation part 113 has, for example, at least one physical button. The brush with an LED light source 100 can be switched to an ON state or an OFF state when the user presses the operation part 113. The operation modes can be changed by the user pressing the operation unit 113. The sensor part 114 includes, for example, an acceleration sensor, a gyro sensor or the like. The accelerometer detects the inclination of the brush with an LED light source 100. For example, by detecting the inclination of the brush with an LED light source 100 by the acceleration sensor, the operation mode may be changed according to the inclination of the brush with an LED light source 100. The above-described linear actuator or an ultrasonic vibrator may be used as the driver 116. The LED driver 118 controls the light emission of the light-emitting element 210 and the light-emitting element 220 in the light-source part 200.

<Structure of Light-Source Part>

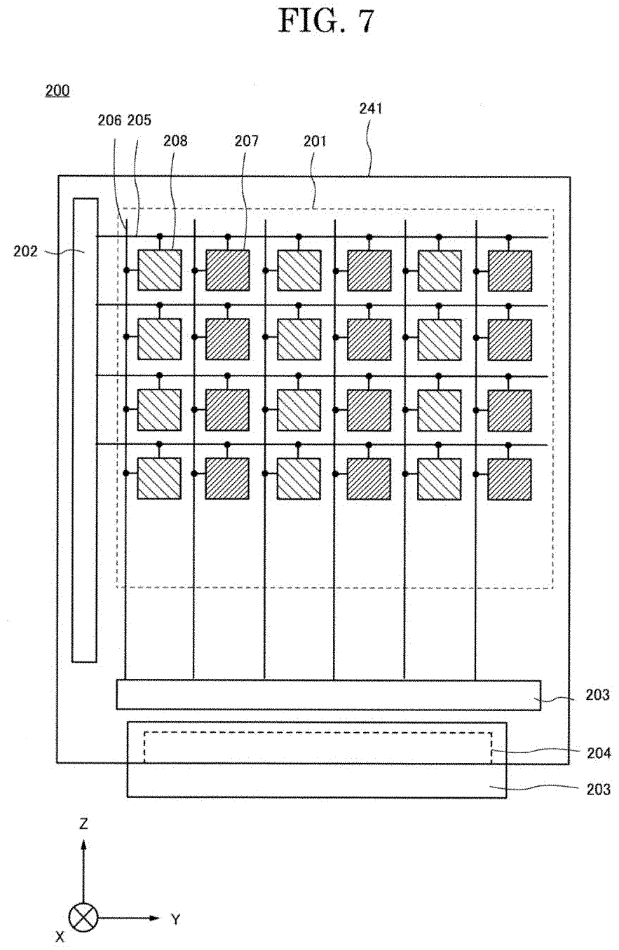

Next, a structure of the light-source part 200 will be described with reference to FIG. 7 to FIG. 9. FIG. 7 shows a plan view of the light-source part 200. The light-source part 200 includes the light-emitting part 201, the scan line drive circuit 202, the signal line drive circuit 203, and the terminal part 204. A pixel 207 provided with the light-emitting element 210 and a pixel 208 provided with the light-emitting element 220 are provided in the light-emitting part 201. In the present embodiment, each of the pixel 207 and the pixel 208 is arranged in a stripe shape along the Z-direction. The pixel 207 is electrically connected to the scan line drive circuit 202 via a scan line 205 and electrically connected to the signal line drive circuit 203 via a signal line 206. Similarly, the pixel 208 is electrically connected to the scan line drive circuit 202 via the scan line 205 and electrically connected to the signal line drive circuit 203 via the signal line 206.

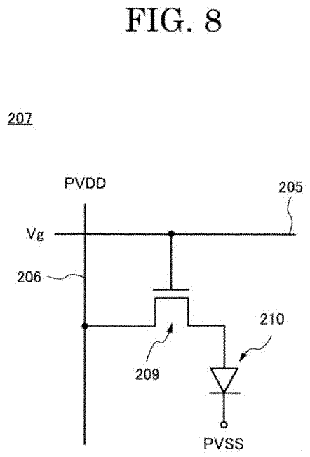

FIG. 8 is a pixel circuit in the pixel 207. The pixel 207 includes the scan line 205, the signal line 206, a transistor 209, and the light-emitting element 210. A gate of the transistor 209 is electrically connected to the scan line 205, one of a source or drain is electrically connected to the signal line 206, and the other of the source or drain is electrically connected to one of the terminals of the light-emitting element 210. The other of the terminals of the light-emitting element 210 is connected to a power line. The scanning line 205 is supplied with a signal Vg, and the transistor 209 is turned on or off according to the signal Vg. The signal line 206 is supplied with a signal PVDD. The power supply line is supplied with PVSS. The pixel circuit of the pixel 208 is the same as the pixel circuit of the pixel 207 except that one of the terminals of the light-emitting element 220 is electrically connected to the other of the source or drain of the transistor 209.

For example, a mini LED ora micro LED is used as the light-emitting element 210 and the light-emitting element 220. An LED that emits ultraviolet light is used as the light-emitting element 210. An LED that emits red light is used as the light-emitting element 220. For example, an AlInGaP/GaP based LED or InGaN/GaN based LED is used as the LED that emits red light. In the case where an InGaN/GaN based LED is used, it is possible to change the emission wavelength according to the current value.

The light-source part 200 can change the light-emitting patterns of the light-emitting element 210 and the light-emitting element 220 according to the operation mode selected by the user. For example, the light-emitting element 210 may be constantly turned on and the light-emitting element 220 may be turned off. The light-emitting element 210 may be always turned off and the light-emitting element 220 may be always turned on. The timing at which the light-emitting element 210 is turned on and the timing at which it is turned off may be the same as or different from the timing at which the light-emitting element 220 is turned on and the timing at which it is turned off.

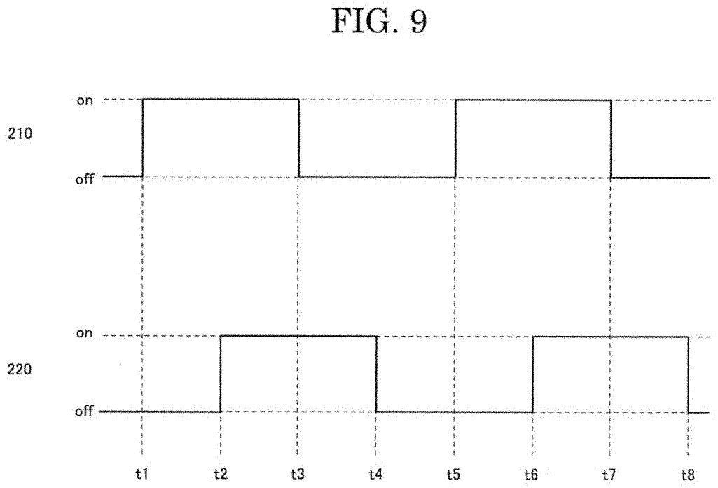

A light-emitting pattern in which the timing at which the light-emitting element 210 is turned on and turned off is different from the timing at which the light-emitting element 220 is turned on and turned off will be described with reference to FIG. 9.

FIG. 9 is a timing chart of light-emitting patterns of the light-emitting element 210 and the light-emitting element 220. In FIG. 9, the light-emitting element 210 is turned on at time t1, and the light-emitting element 220 is turned off. In this case, the brush with an LED light source 100 emits ultraviolet light from the light-emitting element 210. The light-emitting element 220 is turned on at time t2. In this case, the brush with an LED light source 100 emits ultraviolet from the light-emitting element 210 and red light from the light-emitting element 220. The light-emitting element 210 is turned off at time t3 and emits red light from the light-emitting element 220. The light-emitting element 220 is turned off at time t4.

While the present embodiment described the method of driving the light source unit 200 by the active matrix driving method, the light source unit 200 may also be driven by the passive driving method.

### Second Embodiment

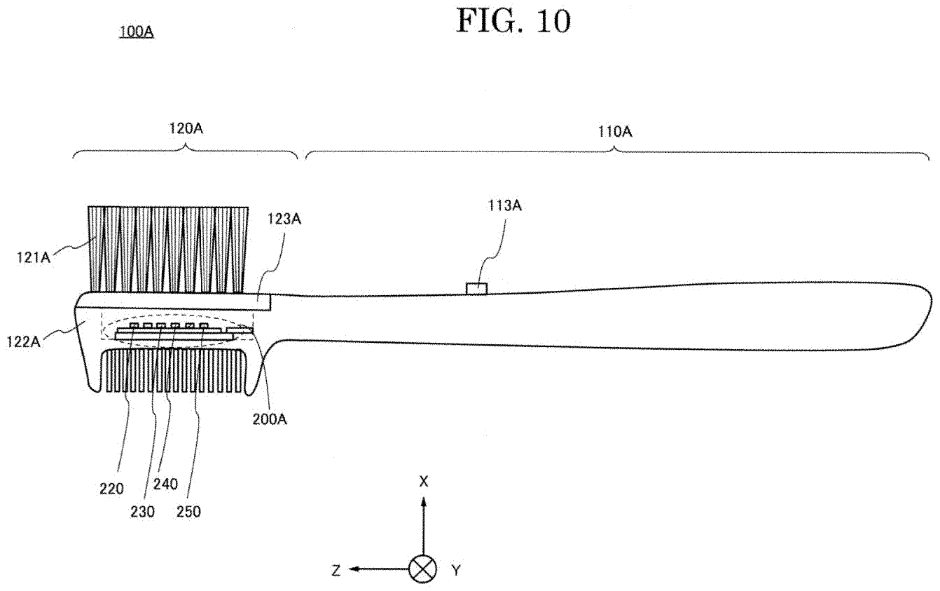

A brush with an LED light source 100A having a partially different structure from the brush with an LED light source 100 shown in the first embodiment will be described with reference to FIG. 10. In the present embodiment, an eyebrow brush will be described as an example of the brush with an LED light source 100A.

FIG. 10 is a cross-sectional view of the brush with an LED light source 100A according to an embodiment of the present invention. In FIG. 10, structures other than a head 120A are the same as that of the first embodiment, and therefore, a detailed illustration thereof is omitted.

A brush part 121A for an eyebrow brush is provided in a support part 123A at the head 120A. A light-source part 200A is provided in a housing part 122A. Instead of the light-emitting element 210, light-emitting elements 230, 240, and 250 are provided in the light-source part 200A. The light-emitting elements 230 to 250 have peaks in the wavelength band of 450 nm to 590 nm. For example, the light-emitting element 230 has a peak in the wavelength band of 565 nm to 590 nm, the light-emitting element 240 has a peak in the wavelength band of 500 nm to 565 nm, and the light-emitting element 250 has a peak in the wavelength band of 450 nm to 485 nm. The light-source part 200A includes a plurality of light-emitting elements 220, a plurality of light-emitting elements 230, a plurality of light-emitting elements 240, and a plurality of light-emitting elements 250. In the light-source part 200A, each of the plurality of light-emitting elements 220, 230, 240, and 250 is arranged in a stripe shape along the Z-direction, for example.

Thus, in the brush with an LED light source 100A, the light-emitting elements 220 to 250 having peaks at different wavelength bands are provided in the light-source part 200A. For example, irradiating skin with the light from the light-emitting element 220 can promote blood circulation. For example, irradiating skin with the light from the light-emitting element 230 can promote melanin excretion. Furthermore, irradiating skin with the light from the light-emitting element 240 can promote fading of skin pigmentation. Also, irradiating skin with the light from the light-emitting element 250 can be promote sterilization of the skin.

In the present embodiment, although an example in which the light-emitting elements 220, 230, 240, and 250 are provided in the light-source part 200A has been described, an embodiment of the present invention is not limited thereto. The light-source part 200A may include, for example, at least two light-emitting elements among the light-emitting elements 220, 230, 240, and 250.

Even in the brush with an LED light source 100A according to the present embodiment, when the brush part 121A is deteriorated or when the support part 123A is damaged, the support part 123A can be easily removed from the fitting part 125. The new support part 123A can be easily attached to the housing part 122. In the brush with an LED light source 100, only the support part 123A needs to be replaced, and the head 120A provided with the light-source part 200A does not need to be replaced, thereby reducing cost.

(Modifications)

Although an embodiment of the present invention has been described, an embodiment of the present invention may be modified in various forms as follows.

The above-described embodiments and the modifications described below can be applied in combination with each other.

(1) In the light-source part 200 shown in FIG. 7, although an example of arranging a plurality of pixels 207A, 208A in a stripe shape has been described, an embodiment of the present invention is not limited thereto. For example, the plurality of pixels 207 and the plurality of pixels 208 may be alternately arranged in a checkered pattern.

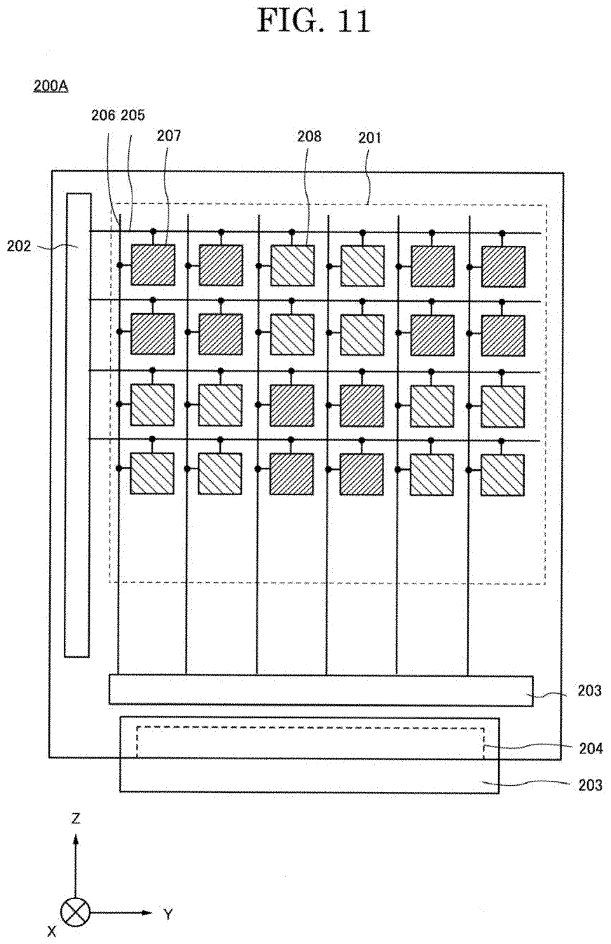

FIG. 11 is an example of the light-source part 200A. The light-source part 200A includes four pixels 207 provided in 2 rows and 2 columns and four pixels 208 provided in 2 rows and 2 columns. Specifically, an example in which four light-emitting elements 210 and four light-emitting elements 220 are alternately arranged is shown. However, the present embodiment is not limited to this, and one light-emitting element 210 and one light-emitting element 220 may be alternately arranged. In the case where the light-emitting element 210 and light-emitting element 220 are arranged in a checkered pattern, a wider range of wavelengths can be obtained as compared with the case where the light-emitting element 210 and the light-emitting element 220 are alternately arranged, and the fluctuation range can be suppressed even when the light-irradiated area fluctuates due to vibrations of the motor or the like.

(2) The lighting rates of the light emitting element 210 and the light emitting element 220 may be changed in the light-source part 200 shown in FIG. 7 and the light-source part 200A shown in FIG. 11. For example, at a predetermined time, the lighting rate of a plurality of light-emitting elements 210 may be 50% and the lighting rate of the plurality of light-emitting elements 220 may be 70%.

The lighting periods of the light emitting element 210 and the light emitting element 220 may be changed in the light-source part 200 shown in FIG. 5 and the light-source part 200A shown in FIG. 11. For example, in the case where the lighting period of the plurality of light-emitting elements 210 is a period T1, the lighting period of the plurality of light-emitting elements 220 may be a period T2 longer than the period T1. In this case, the lighting period of the plurality of light-emitting elements 210 and the lighting period of the plurality of light-emitting elements 220 may overlap or alternate.

In this way, by changing the lighting rate or lighting period of the light-emitting elements 210 and 220 in the light-source parts 200 and 200A, it is possible to irradiate the light emission of the light-emitting elements 210 and 220 at the irradiation intensity and irradiation time desired by the user.

(3) In the brush with an LED light source 100 shown in FIG. 5, although an example in which the support part 123 is formed of a material having light transmittance has been described, an embodiment of the present invention is not limited thereto. In a brush with an LED light source 1006, a support part 123B may be formed of a material that does not have light transmittance (an opaque material).

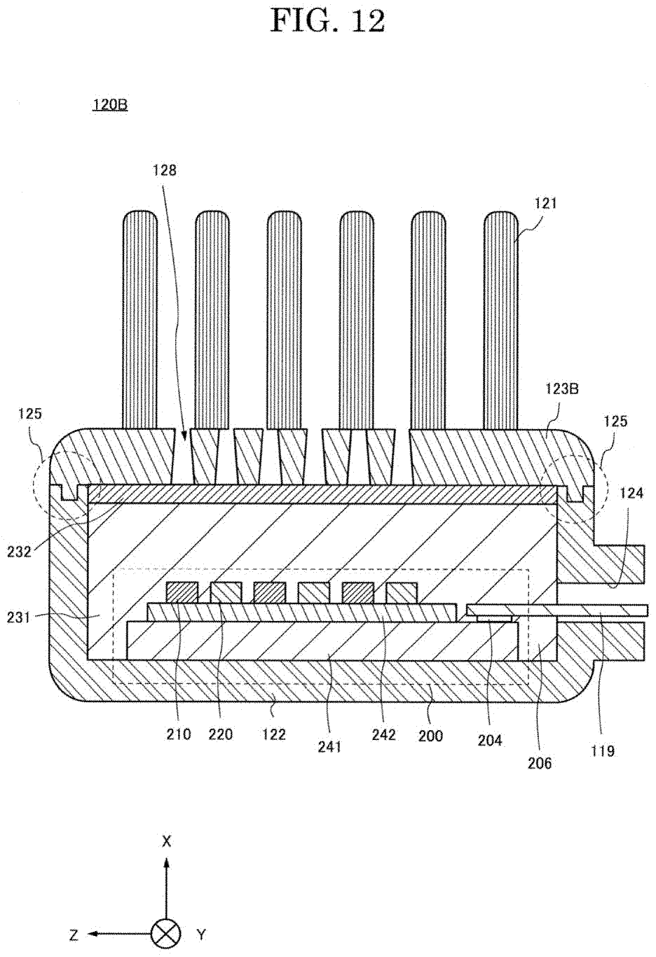

FIG. 12 is a cross-sectional view of a head 120B of the brush with an LED light source 100 according to an embodiment of the present invention. In this case, at least one through hole 128 is preferably provided in the support part 123B to emit light out of the support part 123B when the light-emitting element 210 and the light-emitting element 220 emit light. At least one through hole 128 is provided corresponding to the location where the light-emitting element 210 and the light-emitting element 220 are provided.

The shape when the through hole 128 is viewed from above may be, for example, a circular shape, or a polygonal shape such as a triangular shape, a square shape, and a pentagonal shape. Usually, the particle size of the abrasive particle of toothpaste is distributed in the range of 5 μm to 35 μm. When the abrasive particles are mixed into the through hole 128, the light emitted from the light-emitting element 210 and the light-emitting element 220 is refracted by the abrasive particles, and the light cannot be irradiated to a target location. A hole diameter of the through hole 128 is preferably 5 μm or less to suppress abrasive particles from being mixed into the through hole of the support part 123B. In this specification and the like, the hole diameter of the through hole refers to the smallest circle that can encompass the entire through hole on the surface of the support part 123B. Further, in order to suppress abrasive particles from being mixed into the through hole 128, a resin having light transmittance may be embedded. In this case, the hole diameter of the through hole 128 may be 5 μm to 35 μm.

Since the support part 123 in the brush with an LED light source 100 is removable, it is easy to replace the support part 123 even when abrasive particles penetrate the through hole 128.

(4) In the brush with an LED light source 100 shown in FIG. 12, although an example in which the support part 123B has at least one through hole 128 has been described, an embodiment of the brush is not limited to this. The through hole 128 may be filled with a material 1231 having light transmittance.

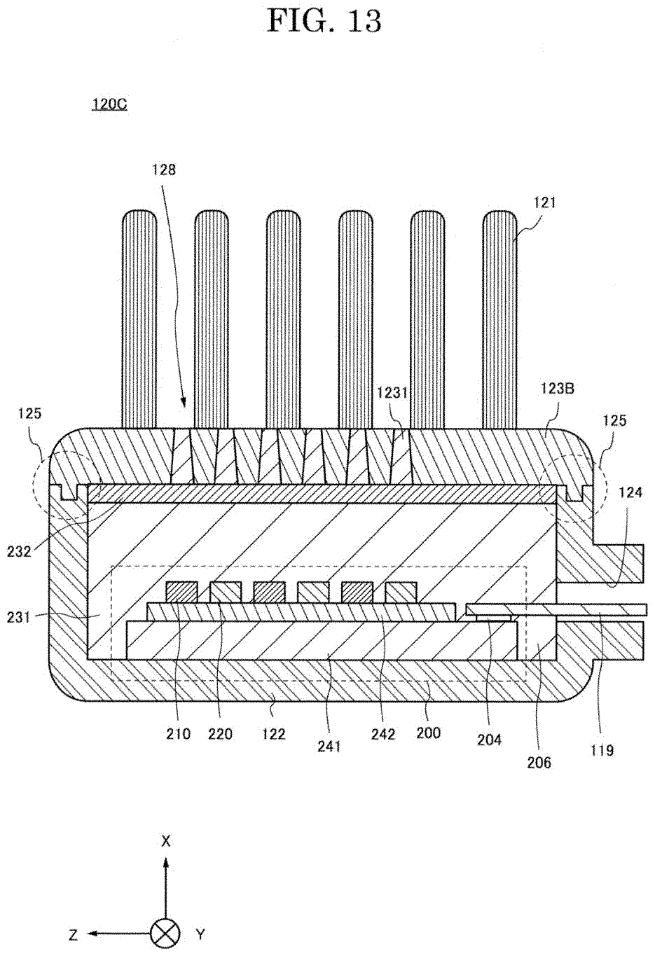

FIG. 13 is a cross-sectional view of a head 120C of the brush with an LED light source 100 according to an embodiment of the present invention. An epoxy resin or an acrylic resin may be used as the material 1231 having light transmittance in the same manner as the material of the support part 123 described in FIG. 5. These materials are materials that are negatively charged in the triboelectric series. By using a material that is negatively charged in the triboelectric series as a material used for the material 1231 having light transmittance, it is possible to suppress the abrasive particles from adhering to the support part 123B, particularly to the material 1231 having light transmittance. As a result, it is possible to suppress the refraction of the light emitted from the light-emitting elements 210 and 220.

In FIG. 13, although an example in which a plurality of through holes 128 is provided in the support part 123B and the through hole 128 is filled with the material 1231 having light transmittance has been described, an embodiment of the present invention is not limited thereto. It is sufficient that the support part 123 has one through hole 128.

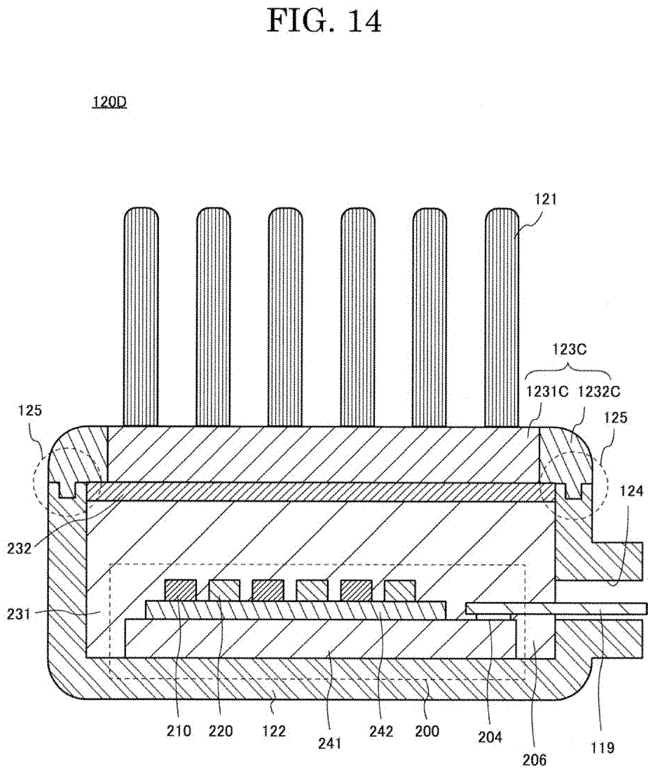

FIG. 14 is a cross-sectional view of a head 120D of the brush with an LED light source 100 according to an embodiment of the present invention. A support part 123C is constituted by a material 1231C having light transmittance and a material 1232C without light transmittance. The material 1231C having light transmittance is provided to overlap the light-source part 200. The brush part 121 is provided on the material 1231C having light transmittance.

(5) In the first embodiment and the second embodiment, although examples in which the brush with LED light sources 100 and 100A are applied to an electric toothbrush and eyebrow brush have been described, the embodiment of the present invention is not limited thereto. The brush with an LED light source in an embodiment of the present invention can be applied to, for example, hairbrushes, makeup brushes, and body brushes.

As described above, the brush with an LED light source 100 according to the embodiment can be applied to various forms. Therefore, the addition, deletion, or design change of components, or the addition, deletion, or condition change of processes as appropriate by those skilled in the art based on the brush with an LED light source 100 described as an embodiment of the present invention are also included in the scope of the present invention as long as they are provided with the gist of the present invention. Each of the embodiments described above can be combined as long as they are provided with the gist of the present invention.

Further, it is understood that, even if the effect is different from those provided by the above-described embodiments, the effect obvious from the description in the specification or easily predicted by persons skilled in the art is apparently derived from the present invention.

Within the scope of the present invention, various modifications and changes understood by persons skilled in the art are also included in the scope of the present invention. For example, the addition, deletion, or design change of components, or the addition, deletion, or condition change of processes as appropriate by those skilled in the art are included in the scope of the present invention as long as they are provided with the gist of the present invention.

What is claimed is:

1. A brush with an LED light source comprising:
a body; and
a head provided at a tip of the body, the head comprising:
a housing part connected to the body;
a support part detachably attached to the housing part;
a brush part formed of a plurality of bristles planted in the support part; and
a light-source part provided inside the housing part,
wherein the light-source part has a first LED for emitting light having a peak in a first wavelength band and a second LED for emitting light having a peak in a second wavelength band different from the first wavelength band.

2. The brush with an LED light source according to claim 1, wherein the first wavelength band is a range of 610 nm to 780 nm, and the second wavelength band is a range of 315 nm to 400 nm.

3. The brush with an LED light source according to claim 1, wherein the first wavelength band is a range of 610 nm to 780 nm, and the second wavelength band is a range of 450 nm to 590 nm.

4. The brush with an LED light source according to claim 1, wherein a first light emission pattern of the first LED and a second light emission pattern of the second LED are different from each other.

5. The brush with an LED light source according to claim 1, wherein the brush part is provided to overlap the light-source part.

6. The brush with an LED light source according to claim 1, wherein at least a region overlapping the first LED and the second LED is made of a material having light transmittance in the support part.

7. The brush with an LED light source according to claim 1, wherein the support part has at least one through hole in a region overlapping the light-source part.

8. The brush with an LED light source according to claim 7, wherein a diameter of the through-hole is 5 μm or less.

9. The brush with an LED light source according to claim 1, further comprising: a light-transmitting resin layer for sealing the top of the light-source part in the housing part.

10. The brush with an LED light source according to claim 9, further comprising:
a glass provided on the resin layer in contact with the support part in the housing part.

11. The brush with an LED light source according to claim 1, wherein the support part is made of epoxy resin or acrylic resin.

12. The brush with an LED light source according to claim 1, wherein the first LED and the second LED are arranged in a stripe pattern in the light source unit.

13. The brush with an LED light source according to claim 1, wherein the first LED and the second LED are alternately arranged in the light source unit.
